(19) United States
(12) Patent Application Publication
(10) Pub. No.: US 2012/0277571 A1
(43) Pub. Date: Nov. 1, 2012

(54) Method For Measuring Trabecular Bone Parameters From MRI Images

(75) Inventors: Gyunggoo Cho, Cheongwon-gun (KR); Namkug Kim, Seoul (KR); June-Goo Lee, Seoul (KR); Youngkyu Song, Cheongwon-gun (KR); Hengjun J Kim, Seoul (KR); Chaejoon Cheong, Cheongwon-gun, Chungcheongbuk-do (KR)

(73) Assignee: KOREA BASIC SCIENCE INSTITUTE, Daejeon (KR)

(21) Appl. No.: 13/094,204

(22) Filed: Apr. 26, 2011

(51) Int. Cl. A61B 5/055; G06K 9/00

(52) U.S. Cl. 600/410; 382/131

(57) ABSTRACT

Disclosed is a method for measuring trabecular bone parameters from MRI images, including: scanning an experimental group with a 3D MRI scanner; segmenting the MRI images to extract bone area and perform skeletonization of the bone area; detecting end-point, joint and branch voxels in the skeleton to analyze bone structure; and measuring trabecular bone parameters based on the result of the structural analysis. The method enables diagnosing osteoporosis.

### BACKGROUND

1. Field

The present disclosure relates to a method for measuring trabecular bone parameters from MRI images.

2. Description of the Related Art

Osteoporosis is a bone disease that leads to a higher risk of fracture, especially in postmenopausal women. In fact, osteoporosis of the hip and osteopenia are experienced by 20% and 34-50%, respectively, of women over 50 years of age and contribute to a 40% lifetime risk of fracture of the hip, radius or spine. Clinically, dual-energy X-ray (DEXA) which measures bone mineral density (BMD) is the current and standard method used to diagnose osteoporosis. According to recent studies, BMD determines approximately 60% of trabecular bone strength. Therefore, it does not sufficient to diagnose osteoporosis using only BMD. In 1994, the World Health Organization (WHO) redefined osteoporosis as a “disease characterized by low bone mass and micro-architectural deterioration causing increased bone fragility.” The measurement of bone density along with micro-architecture measurements including trabecular thickness (TB.Th), trabecular spacing (Tb.S), and trabecular number (TB.N) defines approximately 94% of overall bone strength. Therefore, adequately measuring bone strength requires assessing trabecular bone micro-architecture in addition to BMD. The classical method for evaluating micro-architecture ex vivo was based on histomorphometry results obtained from sections of transiliac bone biopsies. Using this method, the perimeter of the trabeculae is identified on stained sections and its thickness is measured using half of the perimeter. Trabecular bone consists of networks of interconnected plates and rods of 100-150 μm thickness. The image resolution should be compatible with the trabecular thickness to evaluate micro-architecture in vivo. Clinically, there are several imaging modalities, such as peripheral quantitative computed tomography (pQCT), multi-detector computer tomography (MDCT) and micro MRI, which satisfy these criteria. pQCT can only scan peripheral sites in vivo. The achievable resolution of MDCT is insufficient for the quantitative structural analysis of trabecular bone architecture in vivo secondary to limitations on radiation dose. MRI has advantages over MDCT, including being free of ionizing radiation, having a more favorable point spread function (PSF), and the high contrast between bone and marrow. In order to evaluate the micro-architecture of trabecular bone using clinical MRI, 3D high resolution MR images must be obtained. However, the MR images can only be obtained during the limited time that a patient can tolerate remaining still. This MR scan time is determined by resolution as well as the FOV of an image with the same scan parameters. Therefore, high resolution imaging for quantitatively assessing trabecular bone structure in vivo has been limited to peripheral anatomic sites, e.g. the distal radius and tibia.

Most of the fractures caused by osteoporosis occur in the hip and spine; however, it is impossible to obtain MR images at these anatomic sites with a resolution that is comparable to the resolution achievable at more peripheral sites such as the distal radius, tibia or calcaneus. To obtain the micro-architecture parameters of these areas, development of an image processing algorithm which can accurately process the MR trabecular bone images with low resolution is needed.

### SUMMARY

The present disclosure is directed to providing a method for measuring trabecular bone parameters from MRI images.

In one aspect, the present disclosure provides a method for measuring trabecular bone parameters from MRI images, comprising: scanning an experimental group with a 3D MRI scanner; segmenting the MRI images to extract bone area and perform skeletonization of the bone area; detecting end-point, joint and branch voxels in the skeleton to analyze bone structure; and measuring trabecular bone parameters based on the result of the bone structural analysis.

In another aspect, the present disclosure provides a method for diagnosing osteoporosis, comprising: scanning an experimental group with a 3D MRI scanner; segmenting the MRI images to extract bone area and perform skeletonization of the bone area; detecting end-point, joint and branch voxels in the skeleton to analyze bone structure; measuring trabecular bone parameters based on the result of the bone structural analysis; comparing the trabecular bone parameters of a control group with the trabecular bone parameters of the experimental group; and diagnosing osteoporosis based on the result obtained by the comparison.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 shows a flow diagram.

FIG. 2 shows a trabecular bone segmentation result. a) Original images from a MRI machine. b) Re-sampled images into the 512×512×512 matrix size. c) Trabecular bone segmentation images using Otsu's method. d) 2D skeleton images.

FIG. 3 shows zoomed and segmented trabecular bone, its corresponding skeleton and structural analysis result in various resolution images. a) Virtual bone biopsy. b) 3D skeletonization. c) Structural analysis (branch, red line; joint, green sphere; trabecular bone, gray with transparency).

FIG. 4 shows plot of mean and standard deviation of trabecular bone thickness (TB.Th), and the difference between the reference image (65 μm) and the various low resolution images (130, 160, 196, 230, and 265 μm).

### DETAILED DESCRIPTION

Exemplary embodiments now will be described more fully hereinafter with reference to the accompanying drawings, in which exemplary embodiments are shown. The present disclosure may, however, be embodied in many different forms and should not be construed as limited to the exemplary embodiments set forth therein. Rather, these exemplary embodiments are provided so that the present disclosure will be thorough and complete, and will fully convey the scope of the present disclosure to those skilled in the art. In the description, details of well-known features and techniques may be omitted to avoid unnecessarily obscuring the presented embodiments.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second” and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the drawings, like reference numerals denote like elements. The shape, size, regions and the like of the drawing may be exaggerated for clarity.

The present disclosure is to apply a bone segmentation algorithm for images with differing resolutions, to obtain trabecular bone parameter measurements, to evaluate the effect of the resolution on trabecular bone parameters, and to determine an image resolution that is adequately high for accurate structural analysis and can be achieved within a tolerable scan time for patients.

In one exemplary embodiment, the MRI scanning may be performed at 130-230 μm resolution.

When the resolution is lower than 130 μm, the accuracy of measuring the bone parameters may be decreased because of patient's movement. On the other hand, when the resolution is higher than 230 μm, the bone parameters could not be accurately measured due to the limits of spatial resolution and the partial volume effect. The resolution of 230 μm is about twice the typical human trabecular's thickness range (100-150 μm).

In one exemplary embodiment, the segmentation may be carried out by using Otsu's method.

The Otsu's method is used to automatically perform histogram shape-based image thresholding. The algorithm assumes that the image to be thresholded contains two or more classes of pixels (e.g. bone and fat area) then calculates the optimum threshold separating those two or more classes so that their combined spread (intra-class variance) is minimal.

In MRI, mineralized bone is hypointense relative to adjacent tissues because it has low proton density. With the imaging technique applied in the current work, bones essentially have no detectable signal. The MR signal intensity on a voxel of trabecular bone and marrow is attenuated by the independent contributions of the volume proportion of bone and marrow. Therefore, the trabecular bone volume fraction (TBVF) is the ratio of the trabecular bone volume to total bone volume (trabecular bone volume and marrow volume). However, in low resolution MR image acquisition, the partial volume effect (PVE) of MR is amplified by complex factors including spatial resolution, point spread function, the local shape and size of the trabecular bone, material contrast, imaging parameters, etc. Therefore, image segmentation is particularly difficult along the bone-marrow interface secondary to partial volume effects, which are increased in lower resolution images.

Trabecular bone micro-architecture is made up of a complex network of plate and rod structures that are connected by joints. In general, the voxel which contains trabecular bone structure smaller than single voxel size has low intensity level, since voxel intensity is sensitive to spatial resolution, source size and shape, and voxel size. Therefore, the plate, rod and joint structures cannot be correctly evaluated by intensity-based BVF computed from low-resolution MRI. In the present disclosure, the images were thresholded with the Otsu's method. The Otsu's method segments an image into two different classes of pixels, e.g. trabecular bone and marrow, by calculating the optimum threshold separating those two classes so that their combined intra-class variance would be minimal, thus generating more accurate results when the two classes have a comparable number of members. This property of the Otsu's method is appropriate for micro MR imaging of trabecular bone as trabecular bone images consist primarily of two classes of material including trabecular bone and marrow. Therefore, in the present disclosure, segmentation by the Otsu's method was used to evaluate how the MRI resolutions affect the measurement of trabecular bone parameters as measurement of these parameters focused on the segmentation quality of maintaining the bone segmentation region as well as bone structure on low resolution MRI. Contrary to previous TBVF methods that were intended to accurately measure bone mineral density, the present disclosure was intended to evaluate the accuracy of trabecular bone segmentation and bone structural analysis with various image resolutions.

The Otsu's method may be performed after the MRI images are resized to a higher resolution than 130-230 μm resolution.

Table 1 shows voxel size (μm) and Array Dimensions of the Micro MR Imaging Acquisition.

TABLE 1

| Voxel Size (μm) | Array Dimensions |
|---|---|
| 65 | 256 × 256 × 256 |
| 130 | 128 × 128 × 128 |
| 160 | 104 × 104 × 104 |
| 196 | 85 × 85 × 85 |
| 230 | 72 × 72 × 72 |
| 265 | 64 × 64 × 64 |

In one exemplary embodiment, the higher resolution than 130-230 μm resolution may be 65 μm or less, more specifically within the range of 32.5-65 μm.

In one exemplary embodiment, resizing may be performed, including but not limited to, the use of cubic spline interpolation algorithm.

Each voxel on the skeleton can be considered a structural element for bone analysis. To evaluate the trabecular bone structure, the joints and branches of the skeleton were identified. The program tagged all pixel/voxels in a skeleton and then counts all its joints, branches (between joints) and measures branches' average and maximum length.

The voxels are classified into three different categories depending on their 26 neighbors: Wherein the end-point voxels are voxels which have less than 2 neighbors, wherein the joint voxels are voxels which have more than 2 neighbors, wherein the branch voxels are voxels which have exactly 2 neighbors.

In one exemplary embodiment, trabecular bone parameters may be trabecular thickness, bone density, trabecular number, trabecular spacing, local thickness, joint number per unit volume, branch number per unit volume or average branch length.

The trabecular thickness, the bone density, the trabecular number and the trabecular spacing are conventional bone parameters. The local thickness is skeleton-based local trabecular thickness.

The local thickness (slTB.Th) measurement may be implemented to evaluate local bone thickness changes.

The joint number per unit volume, the branch number per unit volume and the average branch length are structural bone parameters based on the result of the bone structural analysis.

Using local thickness, joint number, branch number and the average branch length as new bone parameters is discovered for the first time in the present disclosure.

Table 2 lists the definitions of our trabecular bone parameters.

TABLE 2

| Category | Name | Definition |
|---|---|---|
| Conventional | Trabecular thickness | Average of local thicknesses in |
| Bone | (TB.Th, mm) | every voxel of the skeleton |
| Parameters | Bone density | Bone volume (BV) divided by |
| | (BV/TV, %) | total volume (TV) |
| | Trabecular number | Number of voxels per unit |
| | (TB.N, mm⁻¹) | distance |
| | Trabecular spacing | The distance to the nearest |
| | (TB.S, mm) | skeleton voxel averaged over |
| | | left-right, anterior-posterior, and |
| | | superior-inferior directions |
| Local | Local Thickness | Local thicknesses on each voxel |
| Thickness | (slTB.Th, mm) | of the skeleton of the reference |
| Measurement | | image |
| Structural | Normalized joint | The number of joints in the |
| Analysis | number (nJoint#) | skeleton divided by the number |
| Measurement | | computed from the reference |
| | | images |
| | Normalizedbranch | The number of branches in the |
| | number (nBranch#) | skeleton divided by the number |
| | | computed from the reference |
| | | images |
| | Average branch length | Average length of all branches |
| | (avgBranchLen, mm) | |

In one exemplary embodiment, the experimental group may be patients or suspected patients and the control group may be normal people.

In one exemplary embodiment, the diagnosing comprises predicting that a risk of osteoporosis may be present when the average trabecular thickness of each branch computed by measuring the average branch length and the trabecular thickness of the experimental group is thinner than the average trabecular thickness of control group.

In one exemplary embodiment, the diagnosing comprises predicting that a risk of osteoporosis is present when the local thickness of the experimental group is thinner than the local thickness of control group.

In one exemplary embodiment, the diagnosing comprises predicting that a risk of osteoporosis is present when the joint number per unit volume or the branch number per unit volume of the experimental group is smaller than the joint number per unit volume or the branch number per unit volume of control group.

### EXAMPLES

Examples are described below. The following examples are for illustrative purposes only and not intended to limit the scope of the present disclosure.

### Example 1

### Acquisition of Micro MR Images

Ten bone specimens were extracted from the distal femoral condyle during knee joint replacement procedures at Bundang Seoul National University Hospital.

The specimens were cut using a saw (1×1×1 cm³) and were then fixed in formal in and stored. In preparation for the scan, the bone specimens were defatted, degassed, and immersed in 0.5% (volume percent) gadopentetate-doped water. 3D iso-cubic trabecular bone images of the bone specimens were obtained using a 4.7T Bruker BioSpec MRI scanner with a 40-cm bone size. A 2.5-cm birdcage coil with quadrature detection was used. Factors which impede high resolution 3D imaging of trabecular bone include long scan times and the blurring along the trabecular bone marrow interface caused by the susceptibility difference. Therefore, a 3D fast large-angle spin-echo (FLASE) sequence with a 140° pulse (TR=100 ms and TE=10 ms) was used to overcome these two problems.

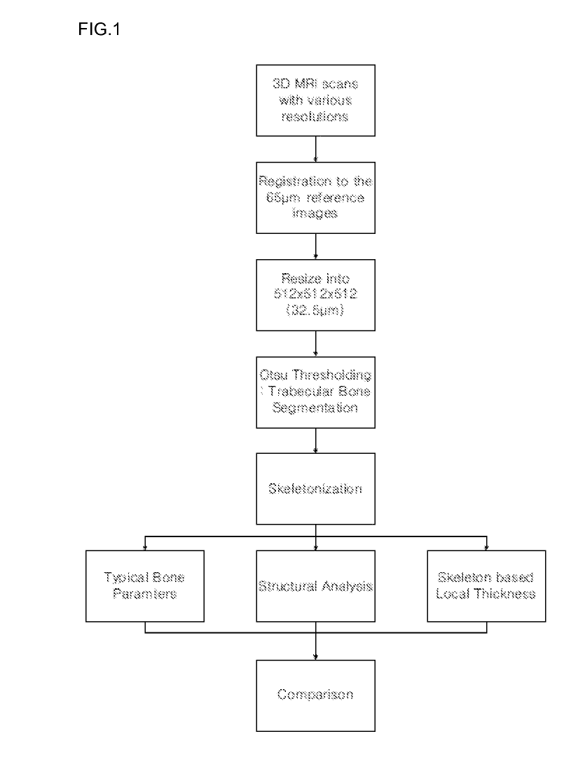

FIG. 1 shows the overall scheme of the present disclosure. First, images of bone specimens acquired with different voxel sizes (130, 160, 196, 230 and 265 μm) were registered to the reference images acquired with a 65 μm voxel size. The images were re-sampled into a 512×512×512 matrix. Using these re-sampled data, trabecular bone was extracted using the Otsu method. Skeletonization was applied after the segmentation, and structural analysis was then performed. Various bone parameters were measured and compared on the segmentation and skeleton. In particular, to compare the voxel-by-voxel local thickness correlation between the reference images and the other images, every local thickness in each voxel of the skeleton of the reference image, was measured and compared. All processing was performed using an in-house program.

### Example 2

### Imaging Preprocessing and Trabecular Bone Segmentation

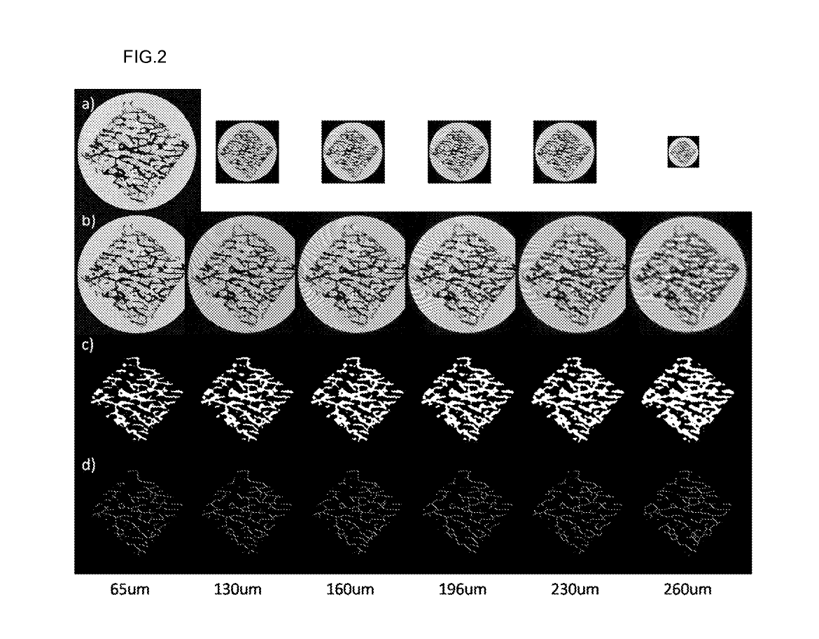

The bone specimens slightly rotated during the acquisition of the images with a 65 μm voxel size secondary to the long acquisition times and strong gradient pulses. The images acquired with larger voxel sizes did not rotate significantly. Therefore, all of the lower resolution images were registered with a high resolution reference image (65 μm cubed iso-cubic voxel size) by rigid registration based on a mean squared difference metric and cubic spline interpolation. After the registration, they were resized as 512×512×512 (iso-cubic voxel resolution: 32.5 μm) using a cubic spline interpolation algorithm. In-house software was developed for these preprocessing steps using the Insight Segmentation and Registration Toolkit (ITK). Trabecular bone extraction from images with various resolutions was performed using the Otsu thresholding method. FIGS. 2a, 2b and 2c show the original images, the re-sampled images, and the segmented images, respectively.

### Example 3

### Trabecular Bone Parameters Evaluation

To evaluate the trabecular bone parameters, skeletonization using a 3D medial surface/axis thinning algorithm was applied to detect the center line of segmented trabecular bone. Each voxel on the skeleton can be considered a structural element for bone analysis. To evaluate the trabecular bone structure, the joints and branches of the skeleton were identified. Structural bone parameters, including joint number (Joint#) and branch number (Branch#) were normalized by those of the reference images due to the large variation of joint and branch numbers between the different specimens. Therefore, normalized joint number (nJoint#), normalized branch number (nBranch#), and average branch length (avgBranchLen) were evaluated. For each voxel along the skeleton, local trabecular bone thickness was measured using a model-independent method. In addition, conventional bone parameters including trabecular thickness (TB.Th), bone density (BV/TV), trabecular number (TB.N), and trabecular spacing (TB.S) were measured based on the skeleton. To calculate TB.Th, local trabecular bone thicknesses on the skeleton were averaged. BV/TV was evaluated by dividing bone volume (BV) by total volume (TV). TB.N, the number of trabeculae per unit distance, was evaluated as BV/TV divided by TB.Th. TB.S was computed as the average distance to the nearest skeleton voxels in left-right, anterior-posterior and superior-inferior directions.

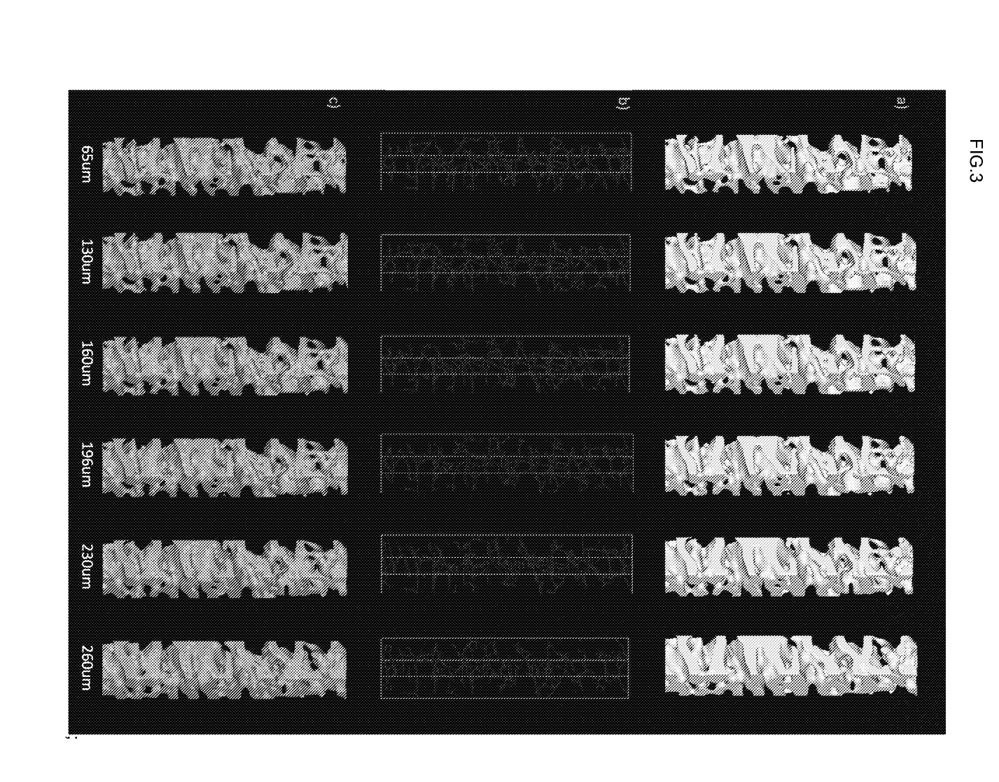

The image processing for a single specimen imaged with each of the different voxel sizes is shown in FIG. 3. For each image resolution, the figure shows 3D surface rendered images of the segmented trabecular bone and its skeletonization. FIG. 3 also identifies the joints and branches along the trabecular network.

### Example 4

### Statistical Analysis

Descriptive statistics of every bone parameter have been computed to compare images with various resolutions. The study population included ten ex vivo specimens. The images with 65 μm voxel size were used as a reference. The bone parameter accuracy between the reference image and other low resolution images was assessed using the Bland-Altman method. In addition, the paired t-test was used to determine the possible presence of a significant measurement difference between the reference image and the other, low resolution images. For validation, Pearson correlation coefficients were computed between local thicknesses measured from the reference images and from the lower resolution images. Statistical significance was defined at an alpha level of 0.05. R v2.10 (R Foundation for Statistical Computing, Vienna, Austria, http://www.R-project.org) was used for these statistical computations.

(1) Conventional Trabecular Bone Parameters

The mean and standard deviation of TB.Th (mm), BV/TV (%), TB.N (mm⁻¹), and TB.S (mm) of the reference images were 0.144±0.032 mm, 29.088±6.683%, 2.030±0.312 mm⁻¹, and 0.466±0.080 mm, respectively. The difference errors between the reference measurements and the bone parameters computed from the lower resolution images were evaluated (Table 3).

Table 3 lists the differences and correlations between conventional bone parameters measured from low resolution Images (130, 160, 196, 230, and 265 μm cubed voxels) and the reference images (65 μm cubed voxels).

TABLE 3

| Voxel | Bone | | | 95% Limits of | | |
|---|---|---|---|---|---|---|
| Size (μm) | parameters | Difference | | agreement* | | |
| Voxel | Bone | P_{ref} − Pₑₛₜ | | 95% Limits of | | r^{†} |
| Size (μm) | parameters | Mean | SD | agreement* | | r^{†} |
| 130 | TB · Th (mm) | −0.008 | 0.006 | −0.020 | 0.005 | 0.986 |
| 130 | BV/TV (%) | −3.697 | 1.359 | −6.351 | −1.022 | 0.982 |
| 130 | TB · N (mm⁻¹) | −0.133 | 0.034 | −0.201 | −0.066 | 0.996 |
| 130 | TB · S (mm) | −0.003 | 0.075 | −0.017 | 0.011 | 0.999 |
| 160 | TB · Th (mm) | −0.016 | 0.007 | −0.030 | −0.003 | 0.984 |
| 160 | BV/TV (%) | −5.775 | 1.379 | −8.477 | −3.073 | 0.982 |
| 160 | TB · N (mm⁻¹) | −0.110 | 0.102 | −0.310 | 0.090 | 0.955 |
| 160 | TB · S (mm) | −0.006 | 0.010 | −0.025 | 0.014 | 0.999 |
| 196 | TB · Th (mm) | −0.033 | 0.011 | −0.054 | −0.011 | 0.943 |
| 196 | BV/TV (%) | −8.945 | 2.298 | −13.449 | −4.440 | 0.960 |
| 196 | TB · N (mm⁻¹) | −0.104 | 0.055 | −0.212 | 0.004 | 0.983 |
| 196 | TB · S (mm) | −0.010 | 0.014 | −0.038 | 0.018 | 0.998 |
| 230 | TB · Th (mm) | −0.047 | 0.012 | −0.070 | −0.024 | 0.932 |
| 230 | BV/TV (%) | −11.780 | −2.910 | −17.483 | −6.077 | 0.942 |
| 230 | TB · N (mm⁻¹) | −0.002 | 0.134 | −0.264 | 0.260 | 0.985 |
| 230 | TB · S (mm) | −0.017 | 0.021 | −0.059 | 0.024 | 0.995 |
| 265 | TB · Th (mm) | −0.074 | 0.019 | −0.111 | −0.037 | 0.819 |
| 265 | BV/TV (%) | −15.443 | 3.456 | −22.217 | −8.670 | 0.929 |
| 265 | TB · N (mm⁻¹) | −0.001 | 0.067 | −0.132 | 0.130 | 0.923 |
| 265 | TB · S (mm) | −0.037 | 0.027 | −0.091 | 0.017 | 0.994 |

Note.
SD = standard deviation,
P_{ref} = one of bone parameters of the reference images (65 μm),
Pₑₛₜ = one of bone parameters of low resolution images (130, 160, 196, 230, and 265 μm).
*Bland-Altman method.
^{†}Pearson correlation coefficients. All correlations were statistically significant.

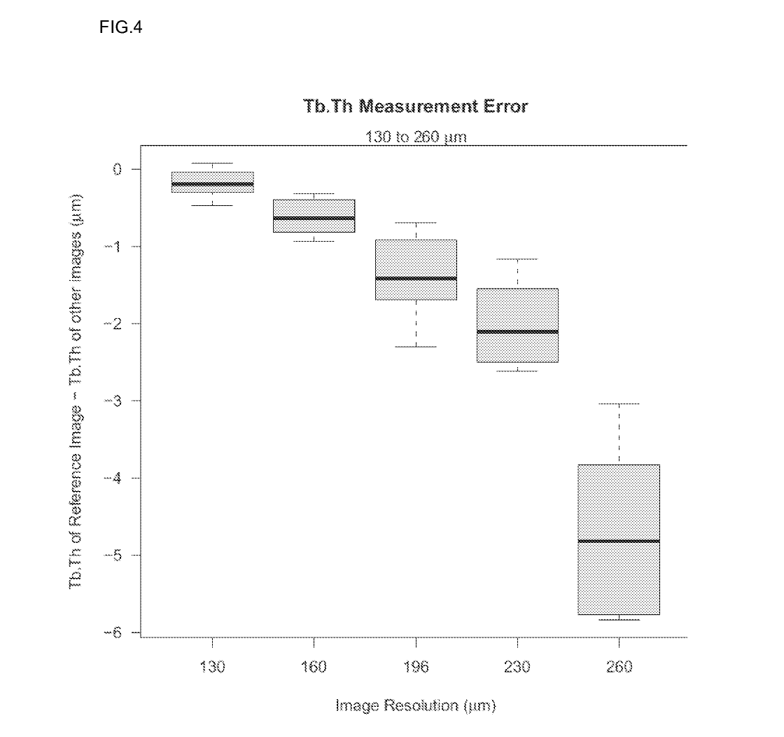

The conventional bone parameters were overestimated in the images acquired with larger voxel sizes. The overestimation is more clearly shown for the parameter TB.Th in FIG. 4, which plots the mean and standard deviation of the difference error as a function of voxel size. However, the correlation coefficients (r) between the parameters computed from the reference images and those computed from the lower resolution images were more than 0.932 if the voxel size was less than 230 μm.

(2) Structural Trabecular Bone Parameters

The mean and standard deviation of structural trabecular bone parameters, including Joint#, Branch#, and avgBranchLen (mm) of the reference image were 2,616.50±907.19, 4,802.90±1,585.44, and 0.417±0.020 mm, respectively. The differences between the structural parameters computed from the reference images and those measured from the lower resolution images were also compared (Table 4).

Table 4 lists the differences and correlations between structural bone parameters measured from low resolution images (130, 160, 196, 230, and 265 μm cubed voxels) and the Reference Images (65 μm cubed voxels).

TABLE 4

| Voxel | Bone | | | 95% Limits of | | |
|---|---|---|---|---|---|---|
| Size (μm) | parameters | Difference | | agreement* | | |
| Voxel | Bone | P_{ref} − Pₑₛₜ. | | 95% Limits of | | r^{†} |
| Size (μm) | parameters | Mean | SD | agreement* | | r^{†} |
| 130 | nJoint# | 0.039 | 0.016 | 0.009 | 0.070 | 0.999 |
| 130 | nBranch# | 0.037 | 0.014 | 0.009 | 0.066 | 0.999 |
| 130 | avgBranchLen | −0.008 | 0.007 | −0.022 | 0.006 | 0.940 |
| | (mm) | | | | | |
| 160 | nJoint# | 0.070 | 0.020 | 0.034 | 0.104 | 0.999 |
| 160 | nBranch# | 0.066 | 0.018 | 0.032 | 0.101 | 0.999 |
| 160 | avgBranchLen | −0.013 | 0.007 | −0.026 | 0.001 | 0.941 |
| | (mm) | | | | | |
| 196 | nJoint# | 0.124 | 0.037 | 0.050 | 0.120 | 0.995 |
| 196 | nBranch# | 0.119 | 0.030 | 0.060 | 0.180 | 0.997 |
| 196 | avgBranchLen | −0.020 | 0.007 | −0.034 | −0.006 | 0.938 |
| | (mm) | | | | | |
| 230 | nJoint# | 0.203 | 0.038 | 0.128 | 0.278 | 0.995 |
| 230 | nBranch# | 0.194 | 0.037 | 0.122 | 0.265 | 0.996 |
| 230 | avgBranchLen | −0.033 | 0.011 | −0.055 | −0.011 | 0.843 |
| | (mm) | | | | | |
| 265 | nJoint# | 0.310 | 0.072 | 0.168 | 0.451 | 0.969 |
| 265 | nBranch# | 0.291 | 0.067 | 0.160 | 0.421 | 0.975 |
| 265 | avgBranchLen | −0.051 | 0.012 | −0.075 | −0.027 | 0.820 |
| | (μm) | | | | | |

Note.
SD = standard deviation,
P_{ref} = one of bone parameters of the reference images (65 μm),
Pₑₛₜ = one of bone parameters of low resolution images (130, 160, 196, 230, and 265 μm).
*Bland-Altman method.
^{†}Pearson correlation coefficients. All correlations were statistically significant.

As voxel size increased, the parameter avgBranchLen was overestimated while nJoint# and nBranch# were underestimated. However, there was good correlation between the parameters computed from the references images and from the lower resolution images (r>0.886) for voxel sizes smaller than 230 μm.

(3) Skeleton-based Local Thickness

0.250±0.034 mm were the mean and standard deviation of the skeleton-based local thickness (slTB.Th) of the reference images. The difference errors between slTB.Th computed from the reference images and from the lower resolution images were evaluated (Table 5).

Table 5 lists the differences and correlations between skeleton-based local thickness measured from low resolution images (130, 160, 196, 230, and 265 μm cubed voxels) and the Reference Images (65 μm cubed voxels).

TABLE 5

| Voxel | Difference | | 95% Limits of | | | | |
|---|---|---|---|---|---|---|---|
| Size (μm) | slTB · Th_{ref} − | | agreement* | | | | Paired |
| Voxel | slTB · Thₑₛₜ | | 95% Limits of | | Pearson Correlation | | t-test |
| Size (μm) | Mean | SD | agreement* | | r | P | p |
| 130 μm | −0.010 | 0.003 | −0.017 | −0.003 | 0.941 ± 0.019 | 0.000 ± 0.000 | 0.000** |
| 160 μm | −0.014 | 0.007 | −0.026 | −0.001 | 0.923 ± 0.021 | 0.000 ± 0.000 | 0.000*** |
| 196 μm | −0.025 | 0.004 | −0.033 | −0.017 | 0.882 ± 0.028 | 0.000 ± 0.000 | 0.000*** |
| 230 μm | −0.035 | 0.005 | −0.044 | −0.026 | 0.833 ± 0.039 | 0.000 ± 0.000 | 0.000*** |
| 265 μm | −0.047 | 0.010 | −0.065 | −0.028 | 0.729 ± 0.073 | 0.000 ± 0.000 | 0.000*** |

Note
Unit: mm
*Bland-Altman method.
**ANOVA test between every correlation study.
***Paired t-test of the higher resolution (the upper row) r-values and the current r-values, i.e. Bonferroni adjustment of significance level alpha = 0.05, was considered to be statistically significant.

The results indicate that slTB.Th was overestimated when measured from the images with larger voxel size. In addition, the ANOVA test and the post-hoc paired t-test showed that every correlation study differed significantly. Table 5 also shows that there was strong correlation between slTB.Th computed from the reference images and from the lower resolution images. As voxel size increased, the correlation coefficients decreased as a result of partial volume effects. However, the correlation coefficients (r) were greater than 0.833 for voxel sizes less than 230 μm.

Basically, as the voxel size became larger, the Otsu segmentation algorithm tended to over-segment bone regions. Therefore, all conventional bone parameters, avgBranchLen as well as the slTB.Th were overestimated, while nJoint# and nBranch# were underestimated, probably due to the loss of structural information in low resolution images. In regard to conventional bone parameters, measurements from high resolution images (65 μm) and from lower resolution images were strongly correlated for BV/TV, TB.N, TB.S, nJoint#, nBranch#, and slTB.Th. However, in the present disclosure, TB.N was sensitive to artifacts of the skeletonization algorithm. The data in Tables 3 and 5 show that the mean difference errors for slTB.Th have smaller absolute values than those for TB.Th, except for measurements from the images with 130 μm cubed voxels. The standard deviations of the difference errors are also mostly smaller for slTB.Th. These results may be secondary to the greater sensitivity of slTB.Th to local structural changes.

The present disclosure has shown that the conventional and structural bone parameters computed on the basis of the images with voxel size up to 230 μm cubed are strongly correlated with measurements from 65 μm cubed voxels (Pearson correlation, r>0.843, p<0.05, Table 4). In addition, measurements of slTB.Th on the basis of larger voxels up to 230 μm cubed are strongly correlated with measurements based on 65 μm cubed voxels (r>0.833, p<0.05). If there were a strong linear correlation between high and low resolution images, linear regression could be incorporated to overcome these kinds of measurement errors. The average slope of ten human bone samples could be used in the correction process.

Traditional study obtained a 3D high resolution image (137×137×410 μm3) of the distal tibia with the scan time of 16.3 min(minutes) and characterized micro-architecture parameters. However, characterization of micro-architectural parameters may be less accurate when image slice thickness (for example, 410 μm) is larger than the thickness of trabecular plates and rods (<150 μm). In the present disclosure, we demonstrated that valid micro-architecture parameters can be measured using the Otsu thresholding algorithm and high resolution isotropic image voxels up to 230 μm cubed. Compared to the voxels of the images acquired by traditional study, 230 μm cubed isotropic voxels would require a decrease in the numbers of phase encoding steps by a factor of 1.68 and an increase in those of slice encoding steps by a factor of 1.78, respectively, to image the same volume of interest. Scan time would thus increase from 16.3 min to 17.3 min. However, the SNR of an image with 230 μm cubed isotropic voxels is 1.58 times greater. The greater SNR can be sacrificed to reduce the imaging time. For example, the steady state signal intensity acquired with a 150 degree flip angle is theoretically 1.58 times less than the signal with the original 140 degree flip angle. With a 156 degree flip angle, TR can be decreased by approximately 30%. The resulting scan time is thus reduced from 17.3 min to 12.1 min while maintaining the SNR at the level in the images acquired by traditional study

While the exemplary embodiments have been shown and described, it will be understood by those skilled in the art that various changes in form and details may be made thereto without departing from the spirit and scope of the present disclosure as defined by the appended claims.

In addition, many modifications can be made to adapt a particular situation or material to the teachings of the present disclosure without departing from the essential scope thereof. Therefore, it is intended that the present disclosure not be limited to the particular exemplary embodiments disclosed as the best mode contemplated for carrying out the present disclosure, but that the present disclosure will include all embodiments falling within the scope of the appended claims.

What is claimed is:

1. A method for measuring trabecular bone parameters from MRI images, comprising:
scanning an experimental group with a 3D MRI scanner;
segmenting the MRI images to extract bone area and perform skeletonization of the bone area;
detecting end-point, joint and branch voxels in the skeleton to analyze bone structure; and
measuring trabecular bone parameters based on the result of the bone structural analysis,
wherein the end-point voxels are voxels which have less than 2 neighbors, wherein the joint voxels are voxels which have more than 2 neighbors, and wherein the branch voxels are voxels which have exactly 2 neighbors.

2. The method for measuring trabecular bone parameters from MRI images according to claim 1, wherein the MRI scanning is performed at 130-230 μm resolution.

3. The method for measuring trabecular bone parameters from MRI images according to claim 1, wherein the segmentation is carried out by using Otsu's method.

4. The method for measuring trabecular bone parameters from MRI images according to claim 3, wherein the Otsu's method is performed after the MRI images are resized to the higher resolution than 130-230 μm resolution.

5. The method for measuring trabecular bone parameters from MRI images according to claim 4, wherein the higher resolution than 130-230 μm resolution is 65 μm or less.

6. The method for measuring trabecular bone parameters from MRI images according to claim 4, wherein the higher resolution than 130-230 μm resolution is 32.5˜65 μm.

7. The method for measuring trabecular bone parameters from MRI images according to claim 4, wherein the resizing is performed by using cubic spline interpolation algorithm.

8. The method for measuring trabecular bone parameters from MRI images according to claim 1, wherein the trabecular bone parameters are trabecular thickness, bone density, trabecular number, trabecular spacing, local thickness, joint number per unit volume, branch number per unit volume or average branch length.

9. A method for diagnosing osteoporosis, comprising:
scanning an experimental group with a 3D MRI scanner;
segmenting the MRI images to extract bone area and perform skeletonization of the bone area;
detecting end-point, joint and branch voxels in the skeleton to analyze bone structure;
measuring trabecular bone parameters based on the result of the bone structural analysis;
comparing the trabecular bone parameters of a control group with the trabecular bone parameters of the experimental group; and
diagnosing osteoporosis based on the result obtained by the comparison,
wherein the end-point voxels are voxels which have less than 2 neighbors, wherein the joint voxels are voxels which have more than 2 neighbors, and wherein the branch voxels are voxels which have exactly 2 neighbors.

10. The method for diagnosing osteoporosis according to claim 9, wherein the MRI scanning is performed at 130-230 μm resolution.

11. The method for diagnosing osteoporosis according to claim 9, wherein the segmentation is carried out by using Otsu's method.

12. The method for diagnosing osteoporosis according to claim 11, wherein the Otsu's method is performed after the MRI images are resized to the higher resolution than 130-230 μm resolution.

13. The method for diagnosing osteoporosis according to claim 12, wherein the higher resolution than 130-230 μm resolution is 65 μm or less.

14. The method for diagnosing osteoporosis according to claim 12, wherein the higher resolution than 130-230 μm resolution is 32.5˜65 μm.

15. The method for diagnosing osteoporosis according to claim 12, wherein the resizing is performed by using cubic spline interpolation algorithm.

16. The method for diagnosing osteoporosis according to claim 9, wherein the trabecular bone parameters are trabecular thickness, bone density, trabecular number, trabecular spacing, local thickness, joint number per unit volume, branch number per unit volume or average branch length.

17. The method for diagnosing osteoporosis according to claim 9, wherein the diagnosing comprises predicting that a risk of osteoporosis is present when the average trabecular thickness of each branch computed by measuring the average branch length and the trabecular thickness of the experimental group is thinner than the average trabecular thickness of control group.

18. The method for diagnosing osteoporosis according to claim 9, wherein the diagnosing comprises predicting that a risk of osteoporosis is present when the local thickness of the experimental group is thinner than the local thickness of control group.

19. The method for diagnosing osteoporosis according to claim 9, wherein the diagnosing comprises predicting that a risk of osteoporosis is present when the joint number per unit volume or the branch number per unit volume of the experimental group is smaller than the joint number per unit volume or the branch number per unit volume of control group.
